(19) United States
(12) Patent Application Publication
(10) Pub. No.: US 2005/0010444 A1
(43) Pub. Date: Jan. 13, 2005

(54) System and method for assisting medical diagnosis using an anatomic system and cause matrix

(76) Inventor: Edwin C. Iliff, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/862,116

(22) Filed: Jun. 4, 2004

Related U.S. Application Data
(60) Provisional application No. 60/476,526, filed on Jun. 6, 2003.

(51) Int. Cl. G06F 17/60; H05G 1/64; A61B 5/00

(52) U.S. Cl. 705/2; 600/300

(57) ABSTRACT

A computerized system and method having a visual display for use in medical diagnosis is disclosed. One embodiment of the system includes a user interface operable to transmit data to the visual display of a computing device, where the user interface substantially simultaneously receives multiple identified attribute values representative of a medical condition of a patient. A data structure is stored in a memory in communication with a processor in the computing device. When the data structure is accessed by the multiple attribute values, it provides a list of one or more selected medical diagnoses of the patient for display via the user interface. The multiple attributes includes at least a first attribute corresponding to a cause of disease and a second attribute corresponding to an anatomic system of the patient.

### PRIORITY

The benefit under 35 U.S.C. § 119(e) of U.S. Provisional Application No. 60/476,526, filed Jun. 6, 2003, entitled “ANATOMIC SYSTEM AND CAUSE MATRIX” is hereby claimed, and this application is incorporated by reference.

### BACKGROUND OF THE INVENTION

1. Field of the Invention

The field of the invention relates to computerized medical diagnostic systems. More particularly, embodiments of the present invention relate to a computerized system and method for diagnosing a patient's medical complaint by utilizing the affected anatomic system and probable cause of the patient's medical complaint.

2. Description of the Related Art

Emergency physicians often have very little time and often even less information about a patient who is brought to the emergency department. Many errors occur because the physician may not consider all of the possible causes of the patient's complaints. A list of the possible diagnoses that a patient can have is called a differential diagnosis.

A few examples will help define the problem. For instance, a patient may be brought in comatose with a high fever. The emergency physician needs to know immediately, sometimes within seconds the causes of coma and particularly the causes of coma that could be caused by infection.

For example, a patient may exhibit a cough and a fever. A logical assumption would be that the patient has an infection (cause) in the respiratory system (anatomic system).

For example, a patient may be brought to the ER in a coma and there may be no history other than that he works in a garage indicating possible carbon monoxide poisoning.

Further, the best testing strategy to distinguish between several possible diagnoses has to be planned in, again, very little time.

What is desired is a way to allow a clinician to start an evaluation based on the limited information that is available. This frequently occurs in emergency medicine, where there are significant constraints on time and information.

### SUMMARY OF CERTAIN INVENTIVE ASPECTS

Embodiments of the invention correspond to a knowledge-based medical software system that is designed to help practicing healthcare workers (particularly emergency medicine physicians) create complete population specific differential diagnoses for their patients and plan the most efficient way to “work up” the patient.

In one embodiment of the present invention there is a computerized system having a visual display for use in medical diagnosis, the system comprising a user interface operable to transmit data to the visual display of a computing device, the user interface configured to substantially simultaneously receive a plurality of identified attribute values representative of a medical condition of a patient, and a data structure stored in a memory in data communication with a processor in the computing device, the data structure being configured, such that when accessed by the plurality of attribute values, to provide a list of one or more selected medical diagnoses of the patient for display via the user interface, wherein the plurality of attributes includes at least a first attribute corresponding to a cause of disease and a second attribute corresponding to an anatomic system of the patient.

The provided data may be identified as being located within the data structure based on the plurality of attribute values. The memory may be included in the computing device, and the memory may be removable. The memory may be updated via data received from a global computing network. The computing device may be portable, and may connect wirelessly to a global computing network.

The user interface of the computerized system may display a representation of at least a portion of the data structure for a user. The representation may include a matrix having symbols indicative of the values of the first attribute along one axis of the matrix and symbols indicative of the values of the second attribute along the other axis of the matrix. The intersection of the identified attribute values on the matrix may identify the list of one or more selected medical diagnoses for the patient. The representation may include symbols indicative of a subclassification of the values of the first attribute and symbols indicative of a subclassification of the values of the second attribute when an input signal is received from the user. The intersection of the identified attribute values on the matrix may identify a list of one or more possible chief complaints for the patient.

The list of one or more selected medical diagnoses for the patient may include a confidence level associated with each diagnosis, and may comprise a rank-ordered differential diagnosis. The anatomic system attribute values may include cardiovascular, respiratory, nervous, digestive/gastrointestinal, ear/nose/throat (ENT), ophthalmologic, gynecologic/obstetric, urologic, blood/hematological, skin/dermatological, bones/orthopedic, and endocrine. The cause of disease attribute values may include allergy, environment, infection, mental, poison, trauma, vascular, genetic, nutritional/metabolic/endocrine, and tumor. Each cause of disease may be subdivided into subcauses of disease. Each anatomic system may be subdivided into anatomic subsystems.

The intersection of the identified attribute values on the matrix may identify a list of one or more potential medical diagnoses for the patient and a list of one or more possible chief complaints for the patient. The list of one or more selected medical diagnoses for the patient may be identified based on the list of one or more potential medical diagnoses for the patient and a user-selected subset of the list of possible chief complaints for the patient when an input signal is received from the user.

The list of one or more selected medical diagnoses for the patient may be identified based on a user-selected subset of the list of possible chief complaints for the patient and one or more criteria when an input signal is received from the user. One of the criteria may comprise the gender of the patient. Another of the criteria may comprise the country or region of the country in which the patient resides. The list of one or more selected medical diagnoses for the patient may be sorted based on the one or more criteria. Another of the criteria may comprise the age of the patient, risk factors of the patient, a travel history of the patient, the race of the patient, or a seriousness of the disease of the patient. The criteria may be dynamically changed.

The data structure may comprise a list of diseases of the human body. A user of the computing device may comprise a physician or other health care professional.

The system may additionally comprise an electronic medical record for the patient, the electronic medical record storing the cause of disease, the anatomic system and at least one of the one or more selected medical diagnoses. The electronic medical record may be provided to at least one of a patient's physician, an emergency room physician, a patient's insurance company, and a managed health organization. The system may additionally comprise an electronic medical record for the patient, the electronic medical record storing the cause of disease, the anatomic system, at least one of the one or more selected medical diagnoses, and the user-selected subset of the list of possible chief complaints. The system may additionally comprise an electronic medical record for the patient, the electronic medical record storing the cause of disease, the anatomic system, at least one of the one or more selected medical diagnoses, and the user-selected subset of the list of possible chief complaints.

The user interface may display a representation of an indicator of the potential seriousness or danger level of the selected medical diagnoses to a healthcare worker. The user interface may display a representation of an indicator of the potential seriousness or danger level of the selected medical diagnoses to the patient. The user interface may display a representation of a work-up based on the selected medical diagnoses, where the work-up may comprise a laboratory test of choice and an imaging modality of choice, and/or where the work-up may comprise a special study of choice and a treatment of choice. The user interface may display a representation of at least one of a history, physical signs, tests, and treatments associated with the patient.

In another embodiment of the present invention there is a computerized method used in medical diagnosis, the method comprising displaying a user interface on a computing device to a user, corresponding a first attribute of a medical condition to a cause of disease and a second attribute of the medical condition to an anatomic system of a patient, substantially simultaneously receiving a plurality of identified attribute values representative of the medical condition of the patient via the interface, accessing, via the computing device, a data structure based on the plurality of attribute values, and providing a list of one or more selected medical diagnoses associated with a diagnosis of the patient to the user interface based on the accessing.

The method may additionally comprise storing the data structure in a memory of the computing device. The method may additionally comprise storing the data structure on a computing network having at least intermittent communication with the computing device. The method may additionally comprise providing the list of the one or more selected medical diagnoses for the patient from the data structure. Displaying the user interface may comprise displaying a representation of at least a portion of the data structure.

In another embodiment of the present invention there is a computerized method used in medical diagnosis, the method comprising displaying a user interface on a computing device to a user; substantially simultaneously receiving a plurality of identified attribute values representative of a medical condition of a patient via the interface, wherein a first attribute of the medical condition corresponds to a cause of disease and a second attribute of the medical condition corresponds to an anatomic system of the patient; accessing, via the computing device, a data structure based on the plurality of attribute values; and providing a list of one or more selected medical diagnoses associated with a diagnosis of the patient to the user interface based on the accessing.

In yet another embodiment of the present invention there is a computerized medical diagnosis system, the system comprising means for displaying a user interface on a computing device to a user; means for substantially simultaneously receiving a plurality of identified attribute values representative of a medical condition of a patient via the interface, wherein a first attribute of the medical condition corresponds to a cause of disease and a second attribute of the medical condition corresponds to an anatomic system of the patient; means for accessing a data structure via the computing device based on the plurality of attribute values; and means for providing a list of one or more selected medical diagnoses associated with a diagnosis of the patient to the user interface based on the accessing.

The system may additionally comprise means for storing the data structure in a memory of the computing device. The system may additionally comprise means for storing the data structure on a computing network having at least intermittent communication with the computing device. The system may additionally comprise means for providing the list of the one or more selected medical diagnoses for the patient from the data structure. The means for displaying the user interface may comprise means for displaying a representation of at least a portion of the data structure.

### BRIEF DESCRIPTION OF THE DRAWINGS

FIG. 1 is a block diagram of one embodiment of a diagnostic system incorporating an embodiment of a matrix application.

FIG. 2 is a diagram of one embodiment of a top level array of cells utilized in the matrix application of FIG. 1.

FIG. 3 is a diagram of one embodiment of a detailed level array of cells utilized in the matrix application of FIG. 1.

FIG. 4 is a diagram of another embodiment of a detailed level array of cells utilized in the matrix application of FIG. 1.

FIG. 5 is one embodiment of an exemplary screen display of a patient information tab screen of the matrix application of FIG. 1.

FIG. 6 is one embodiment of an exemplary screen display of a matrix tab screen of the matrix application of FIG. 1.

FIG. 7 is an exemplary screen display of the matrix tab screen of FIG. 6 showing a selected cell in an array portion of the screen, possible chief complaints in a possible chief complaint portion of the screen, and a list of possible diagnoses in a disease portion of the screen.

FIG. 8 is an exemplary screen display of the matrix tab screen of FIG. 6 showing a selected chief complaint in a selected chief complaint portion of the screen, and a corresponding list of possible diagnoses in the disease portion of the screen.

FIGS. 9a and 9b are a flowchart of one embodiment of a process of operation for the system shown in FIG. 1.

FIG. 10 is an exemplary screen display of the matrix tab screen of FIG. 6 showing two selected chief complaints in the selected chief complaint portion of the screen and a corresponding list of possible diagnoses in the disease portion of the screen.

FIG. 11 is an exemplary screen display of the matrix tab screen of FIG. 6 showing a list of ranked possible diagnoses in the disease portion of the screen after a diagnose button is activated on the screen.

FIG. 12 is a diagram illustrating exemplary operation of a portion of the matrix application after a cell on the array is selected, such as on the array of FIG. 6.

FIG. 13 is a diagram illustrating exemplary operation of a portion of the matrix application after a column on the array is selected, such as on the array of FIG. 6.

FIG. 14 is a diagram illustrating exemplary operation of a portion of the matrix application after a row on the array is selected, such as on the array of FIG. 6.

FIG. 15 is a diagram illustrating exemplary operation of a portion of the matrix application after the diagnose button shown in FIG. 6 is activated.

FIG. 16 is a diagram illustrating exemplary operation of a portion of the matrix application after the diagnose button shown in FIG. 6 is activated and data from the patient information tab shown in FIG. 5 is applied.

FIG. 17 is an exemplary screen display of information about a particular disease selected from the ranked possible diagnoses.

FIG. 18 is another embodiment of an exemplary screen display of the matrix tab screen of the matrix application of FIG. 1.

FIG. 19 is one embodiment of an exemplary screen display of a work-up tab screen having a lab test tab selected of the matrix application of FIG. 1.

FIG. 20 is one embodiment of an exemplary screen display of a work-up tab screen having an imaging tab selected of the matrix application of FIG. 1.

### DETAILED DESCRIPTION OF CERTAIN INVENTIVE EMBODIMENTS

The following description presents certain specific embodiments of the present invention. However, the present invention may be embodied in a multitude of different ways as defined and covered by the claims. In this description, reference is made to the drawings wherein like parts are designated with like numerals throughout.

In certain embodiments, a goal of the Matrix system is to provide very rapid access to the information that a clinician needs at the bedside. The Matrix answers the question “What could my patient have?” And then the question, “How do I best plan the work up of the patient?”

A goal of various embodiments, termed “the Matrix system” or “matrix” is to provide a “population” specific differential diagnosis. Population specific refers to the age, sex, race, location, work place, travel history of the patient, etc. The Matrix allows the user to look at disease information by anatomic system the disease may occur in, the possible causes of a problem, the chief complaint, by age groups, by gender, etc. In fact, the Matrix allows the user to dynamically change the sorting parameters themselves.

It is fast, easy to use and structures the information in such a way as to be meaningful to a clinician at the bedside. The Matrix application can adjust to the sophistication of the user. For example, there are versions for Emergency Medical Technicians (EMTs), Nurses, Primary care physicians, Emergency Physicians, and Specialists.

Platform

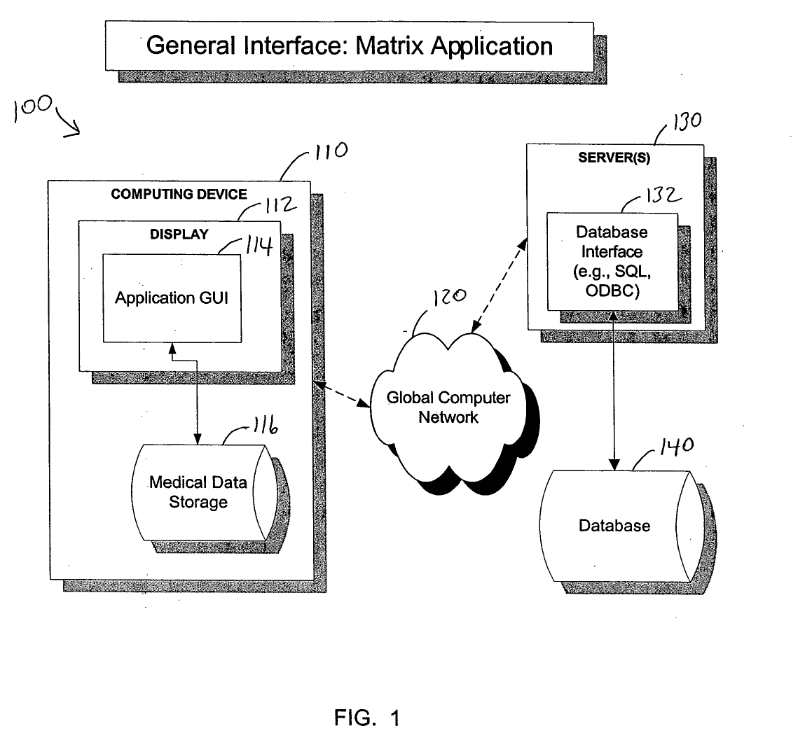

Referring to FIG. 1, one embodiment of a diagnostic system 100 incorporating an embodiment of a matrix application 114 will be described. The Matrix 114 can be embodied as an application graphical user interface (GUI). In various embodiments, the application GUI 114 runs on a handheld computing device 110 or other portable computing device such as a Palm, Pocket personal computer (PC), Linux based handheld, Tablet PC, or PC having a display 112. The application GUI 114 may include a database interface to the medical data storage 116 of the computing device. One embodiment of the Matrix application was developed using Microsoft Visual Basic version 6.0.

The Matrix 114 may also be optionally configured to connect to the Internet or other global computer network 120 either directly or via wireless or satellite technology. When connected to a global computer network, such as the Internet, the Matrix 114 can communicate with a central or distributed database 140 via one or more servers 130 having a database interface 132, such as structured query language (SQL) or open database connectivity (ODBC). Of course, data communication networks other than global networks may be provisioned.

Medical Data

The Matrix 114 (FIG. 1) contains its own database of medical data in the medical data storage 116, so that it can run independently of any other computer. In one embodiment, the database includes several tables, such as a chief complaint table and a disease table. A web site may optionally provide updates on new disease information or about new laboratory tests, special studies, imaging modality of choice and treatment of choice. In another embodiment, the Matrix 114 runs only when connected to a central or distributed database.

In addition, the Matrix 114 can optionally be linked to the global network 120 to allow instantaneous reporting of and downloading information about, for example, possible epidemics or the use of weapons of mass destruction (WMD).

Patents

Certain related concepts based on using a two-dimensional array, are described in Applicant's U.S. Pat. No. 5,594,638, which is hereby incorporated by reference. U.S. Pat. Nos. 5,935,060, 6,022,315, 6,234,964 and 6,524,241 are also hereby incorporated by reference.

Matrix Concept

One Matrix concept is based on the geometric metaphor of a two-dimensional array in which the causes of disease are listed on a vertical axis and the anatomy in which they occur along the horizontal axis. Diseases can then be classified by a given Cause and a given Anatomy, and listed at the intersection of a Cause row and an Anatomy column. In one embodiment, the cause axis and the anatomy axis could be interchanged.

If more detail is desired along either the cause or anatomy axes, any cell can be “exploded” into its own sub-matrix, with more detail along either axis. A computer program organized using this concept allows a user to “browse” the domains of cause and anatomy, to change levels along either axis as needed, and to “drill down” to see any convenient detail of information.

Matrix Example

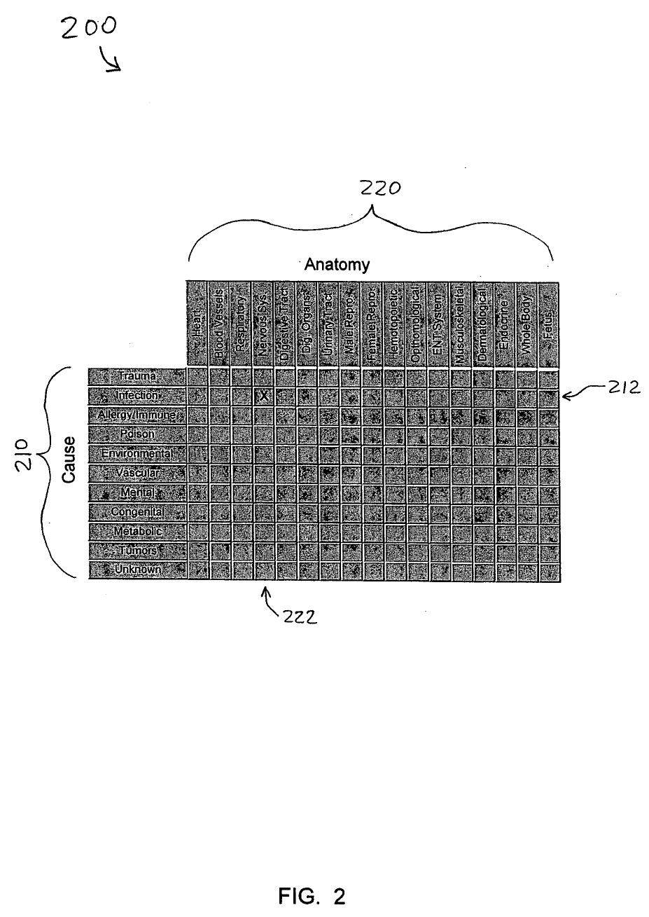

Referring to FIG. 2, an illustration of one embodiment of a portion of a matrix software implementation is shown. The figure shows an exemplary top-level table 200 of cells for all Causes 210 and Anatomies 220, and an X has been inserted in one of the cells to indicate an infection 212 of the nervous system 222 that is of current interest to the user. The user might wish to list all of the diseases found at the X cell, which, in this example, are all diseases caused by an infection of the nervous system, such as encephalitis, meningitis, rabies, tetanus, etc.

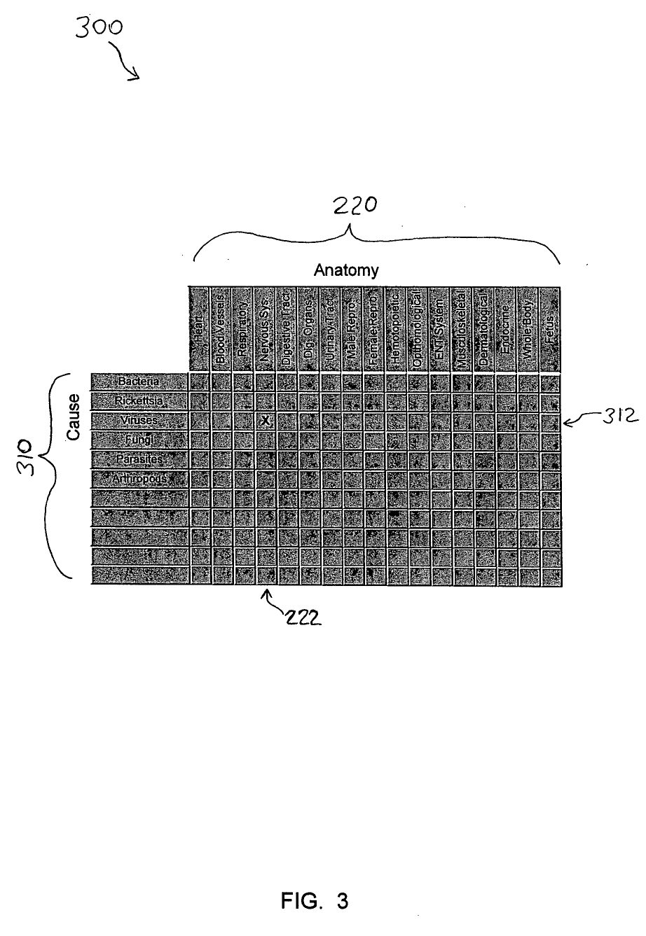

Alternatively, the user might be interested in seeing a more detailed level of the cause axis for infection and obtain an array 300 as shown in FIG. 3. Any of various selection methods, such as clicking, can be used to indicate to the Matrix GUI 114 that a more detailed level of the cause axis is requested. In FIG. 3, the top level cause axis 210 of FIG. 2 is replaced by a second level cause axis corresponding to infection 310. Referring to FIG. 3, at this level, the X in the cell represents all viral infections 312 of the nervous system 222, such as meningitis or herpes encephalitis.

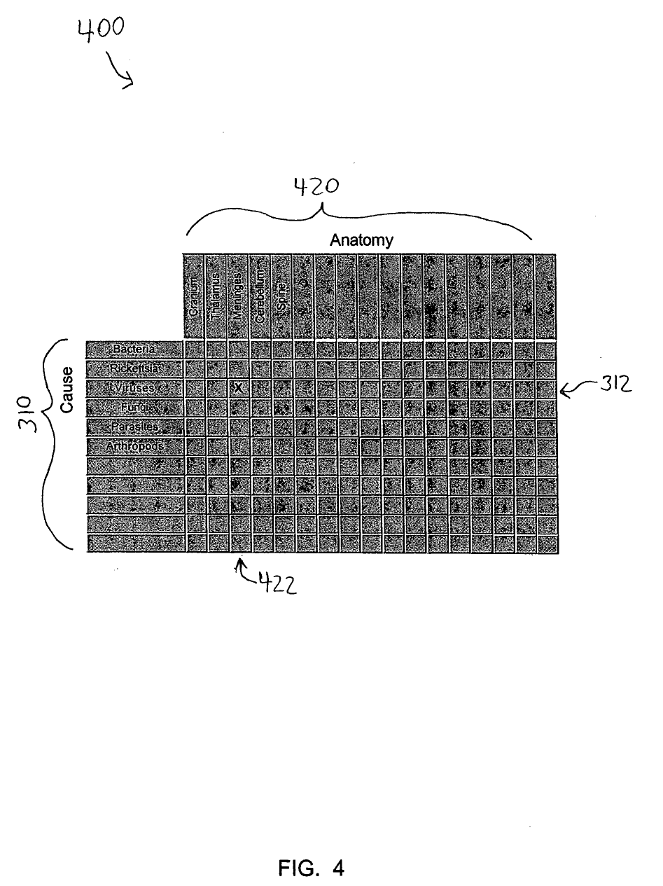

Further, a user might want to see more detail for the nervous system along the anatomic axis and obtain an array 400, which can be subdivided into organs, tissues, cells, etc. as shown in FIG. 4. In FIG. 4, the top level anatomy axis 220 of FIGS. 2 and 3 is replaced by a second level anatomy axis corresponding to nervous system 420. Referring to FIG. 4, at this level, the X represents a viral infection 312 of the meninges 422, i.e., a diagnosis of viral meningitis.

Note that this and other figures show but certain embodiments of the invention and that use of the term “Matrix” to describe these embodiments does not in any way limit the inventor's scope of protection.

Effect of Time

As described above, in various embodiments, the Cause/Anatomy Matrix represents a given moment in time in a patient's illness. As a disease progresses and evolves in the patient, it typically moves into or affects other parts and levels of the anatomy, causing other diseases as well as external symptoms. If the progress of a disease is plotted over time, as a series of matrices, it generates a “Time Cube” (e.g., a parallelepiped) and the disease can now be seen more usefully as a succession of patient health states over time. Needless to say, the assistance of a computer is necessary to generate and maintain such a detailed mathematical object; but its diagnostic benefits are well worth it.

The “Time Cube” represents a Cause/Effect sequence as it moves through the patient's anatomy over time. It is a superb summation of all of the patient's past history that is stored in the patient's medical record. It allows very useful analyses to be performed.

Matrix Program Embodiments

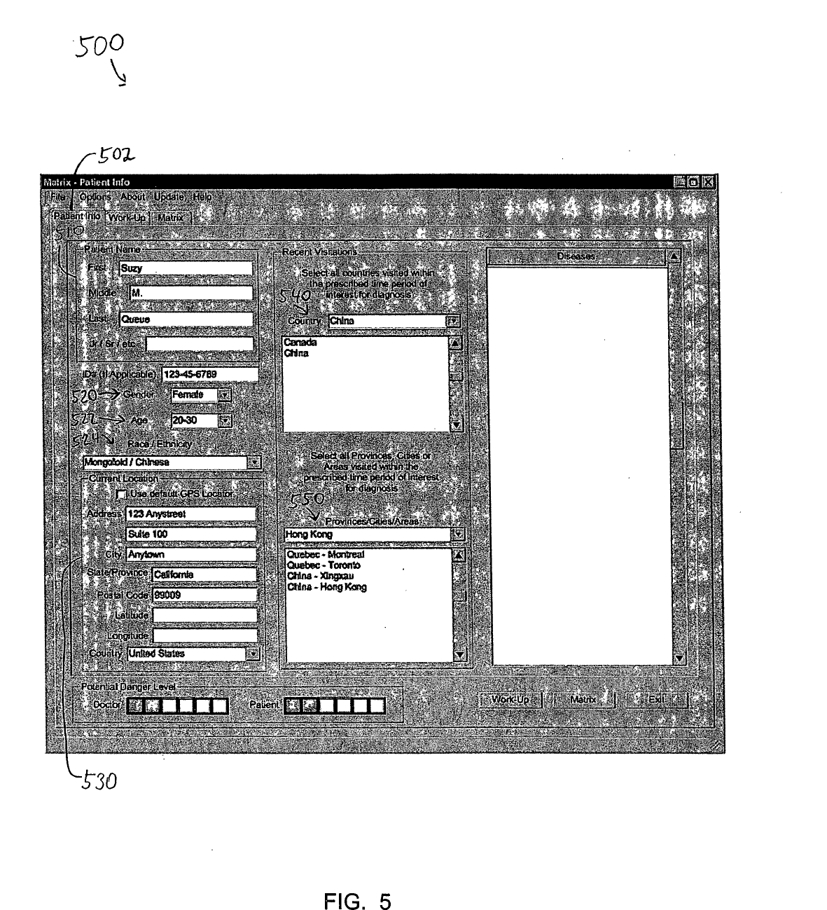

Before using the actual Matrix itself, the application program 114 (FIG. 1) may allow, in certain embodiments, for the input of patient specific information. Referring to FIG. 5, an exemplary patient information screen 500 having a patient information tab 502 is shown. Along with patient name 510, age 522 and gender 520, in one embodiment, the patient information screen allows for ethnicity 524, current location 530 and travel information 540/550 within the timeframe of interest. For example, the application requests information about countries 540, areas and cities 550 visited within the period of interest for diagnosis. This, coupled with age and gender, provide additional tools which are valuable in sorting out certain kinds of diseases that are population or location specific in their frequency of occurrence.

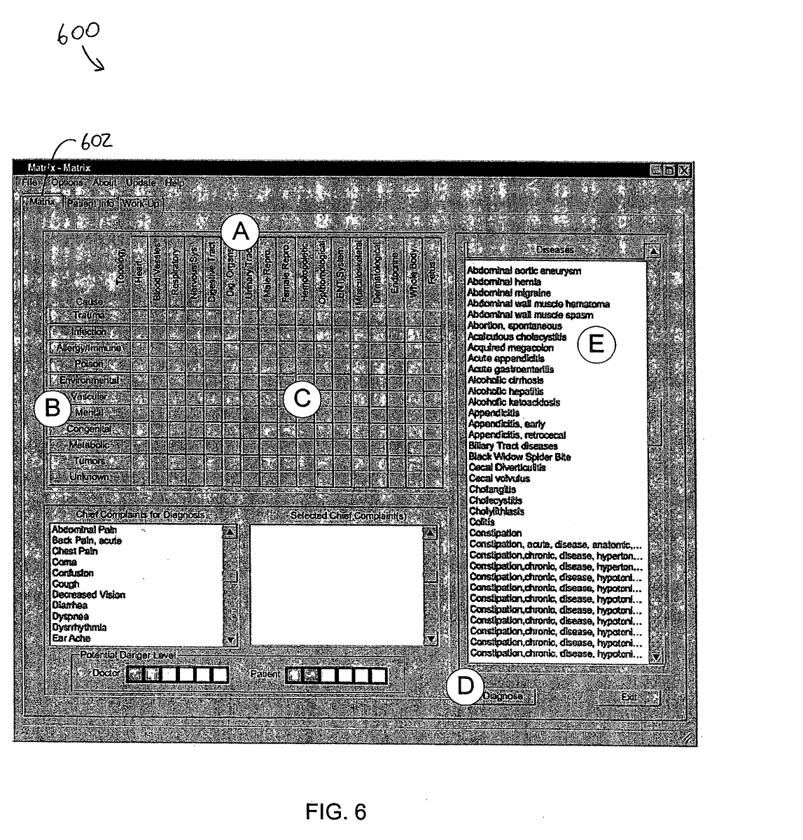

Referring to FIG. 6, in one embodiment, a Main Matrix tab 602 of Matrix screen 600 shows a grid of Cause of Diseases (e.g., trauma, infection, allergy or immune or inflammation, poisoning, environmental, vascular, mental, congenital, metabolic or endocrine, tumors, and unknown) (as shown at area B) versus Anatomic System (e.g., heart, blood vessels, respiratory, nervous system, digestive tract, digestive organs, urinary tract, male reproductive, female reproductive, hemotopoietic, opthomological, ear-nose-throat (ENT) system, musculoskeletal, dermatological, endocrine, whole body, and fetus) (as shown in area A). The cells of the grid (as shown at area C) represent a list of diseases of the given anatomy that are associated with the given cause.

The lower left of the main screen displays the chief complaints (CC) referable to the diseases selected by clicking on the Matrix. The right half of the main screen shows the current disease list (and after a Diagnose operation is selected, the Differential Diagnosis) as shown at area E.

User Interaction with the System

Further referring to FIG. 6, on the embodied Main Matrix tab 602 of the Matrix screen 600, the user can click any Anatomic System heading (area A) to display all of the diseases of (say) the Respiratory System. Similarly, the user can click a Disease Cause (area B) to display all of the diseases caused by (say) Infection. Or the user can click any cell at the intersection (area C) to display all of the diseases of (say) the Respiratory tract caused by Infection.

The user may click on the chief complaint referable to each anatomic system, for example, and cause a differential diagnosis to be displayed in area E.

In addition, the user can click on several of the chief complaints and see the diseases that include n or n−1 of the chief complaints. A Diagnose button or selector D will be described hereinbelow.

Layered Matrix Format

One interesting attribute of the Matrix application 114 (FIG. 1) is the manner in which it organizes medical data into layers of detail. This layering helps to match the “granularity” of the data to the current needs of the user, from physician to nurse to patient. In many cases, each cell of a matrix is itself a matrix, which “blows up” to display terminal data or still other matrices at its cells.

Thus on the Main Matrix tab 602 (FIG. 6), the user sees Anatomy and Cause. Clicking the cell at each intersection (Anatomy, Cause) lists either the relevant diseases (with a single click, in one embodiment, or with a double click in another), it allows the user to “drill down” to another layer of detail. For example, a double click on the cell at the intersection of Respiratory (anatomic system), and Infection expands to show a sub-matrix that lists the different types of organisms that can cause infection (bacteria, viruses, mycoplasma, fungi, etc.) on the ordinate and the first sub-layer of the respiratory tract along the top.

In general, a double click takes the user to more detailed or granular information. The clinical user might then ask to see “all infections caused by viruses in the bronchioles of the lung”.

As the user “drills down” to lower levels of detail, other areas of the screen show the appropriate detail. Thus, the differential diagnosis lists at area E change depending on the detail level.

The number of nested layers that are provided depends, of course, on the user as well as the specific topic. For a primary care physician, three levels of anatomic and cause data are typically sufficient. Specialty versions of Matrix can go to the depth appropriate to the specialty.

Selection of Chief Complaint

In one embodiment, there are two ways to have the chief complaint window operate. The most general way is what is termed the “backwards” methodology in which the Matrix application 114 (FIG. 1) finds all of the disease caused by infection in the respiratory system, then looks at the chief complaints associated with those diseases and lists them. The user sees a display of chief complaints such as abdominal pain. Abdominal pain can be a complaint of a patient with pneumonia, for example.

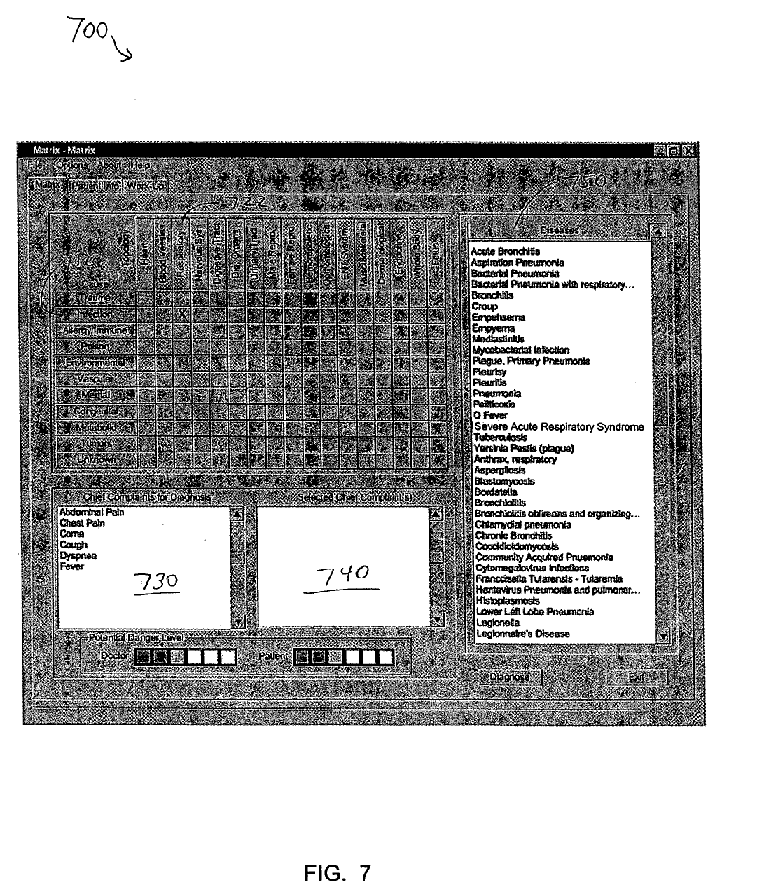

Referring to FIG. 7, an exemplary Matrix screen 700 is illustrated. The matrix disease window 750 (area E, FIG. 6) contains a list of diseases in alphabetical order which are only associated with respiratory 722 infection 712. As information is input, the list of diseases adjusts itself to promote those diseases to the top of the list that “best fit” the profile of complaints and patient information given by the physician. Chief complaints corresponding to the respiratory infection cell are shown in a chief complaints area or window 730. A selected chief complaints area or window 740 is empty in the example of FIG. 7.

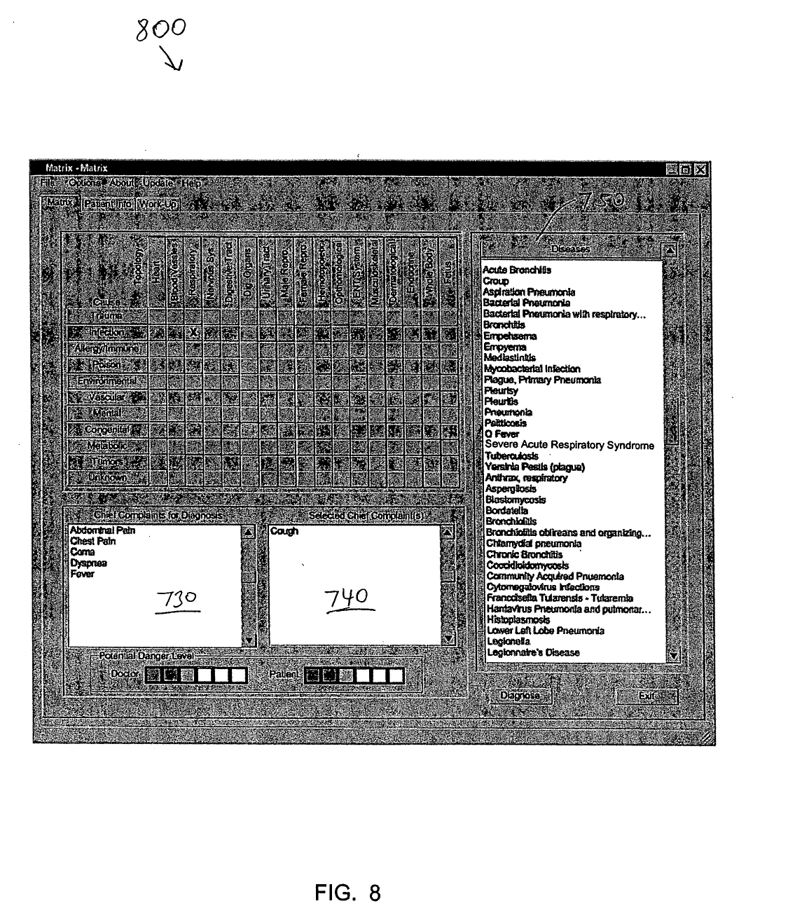

Referring to the exemplary screen 800 shown in FIG. 8, once “Cough” has been selected from the chief complaints window 730, for example, it is displayed in the selected chief complaints window 740. The Matrix 114 then re-assesses its diseases in the list 750 and in this instance promotes “Croup” to the second position, as it now becomes a more likely candidate for the diagnosis. This process is more fully described in the “Pick List Function” section below.

Chief Complaint Window Generalized

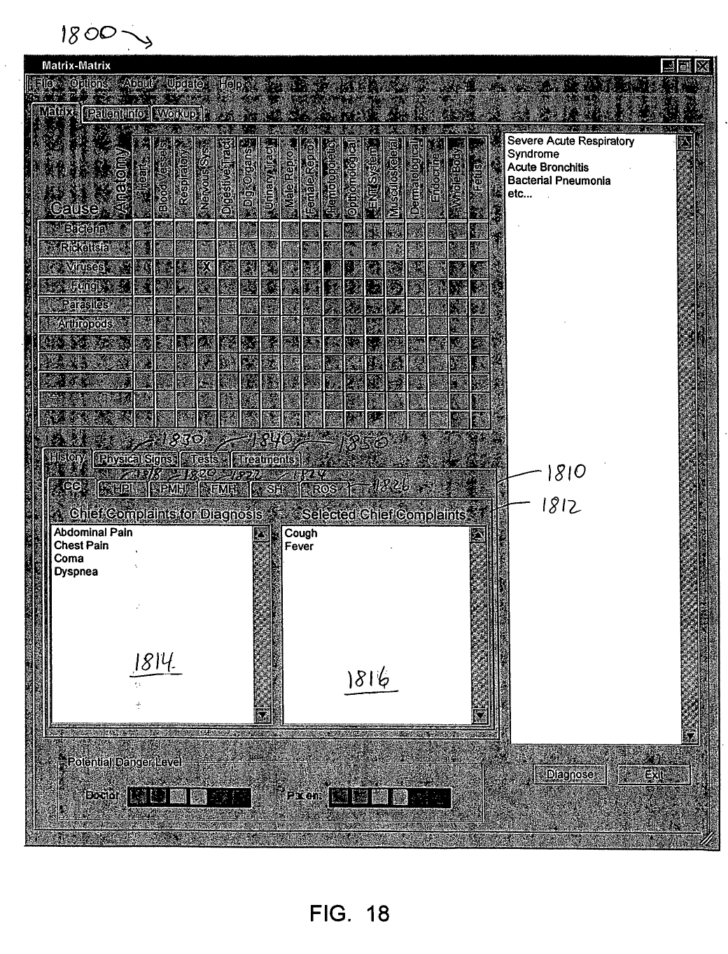

In one embodiment, the area for listing the chief complaints 730 (FIG. 7) and the selected chief complaints 740 is generalized to encompass other aspects of a medical caregiver's work-up. Referring to FIG. 18, an exemplary screen display 1800 showing the matrix tab window 602 (FIG. 6) for the matrix application will be described. The lower left portion of the exemplary screen display 1800 includes multiple tabs for a patient history 1810, physical signs 1830, tests 1840 and treatments 1850 associated with the particular patient. Under the patient history tab 1810 are a series of lower level tabs including a chief complaint (CC) tab 1812, a history of present illness (HPI) tab 1818, a past medical history (PMH) tab 1820, a family medical history (FMH) tab 1822, a social history (SH) tab 1824, and a review of systems (ROS) tab 1826. The CC tab 1812 can include a display window 1814 of possible chief complaints for diagnosis, and a display window 1816 of selected chief complaints. The physical signs tab 1830 can include lower level tabs such vital signs (VS) and physical examination findings (PE), e.g., wheezing on auscultation of the chest. The tests tab 1840 can include lower level tabs such as laboratory test of choice (LTC), special study of choice (SSC), ancillary study of choice (ASC), and imaging modality of choice (IMC). The treatments tab 1850 can include a lower level tab such as treatment of choice (TOC). Of course, other tabs and displays are contemplated. The data displayed in the tabs is stored in various fields in the database, and can be initially entered into the database based on past consultations and the current consultation, completed forms associated with the patient, lab and test results, data transferred in from other sources, and so forth.

Pick List Function

The pick list function allows the user to select more than one chief complaint, symptom, vital sign, physical sign, etc., and then tell the system to “diagnose.”

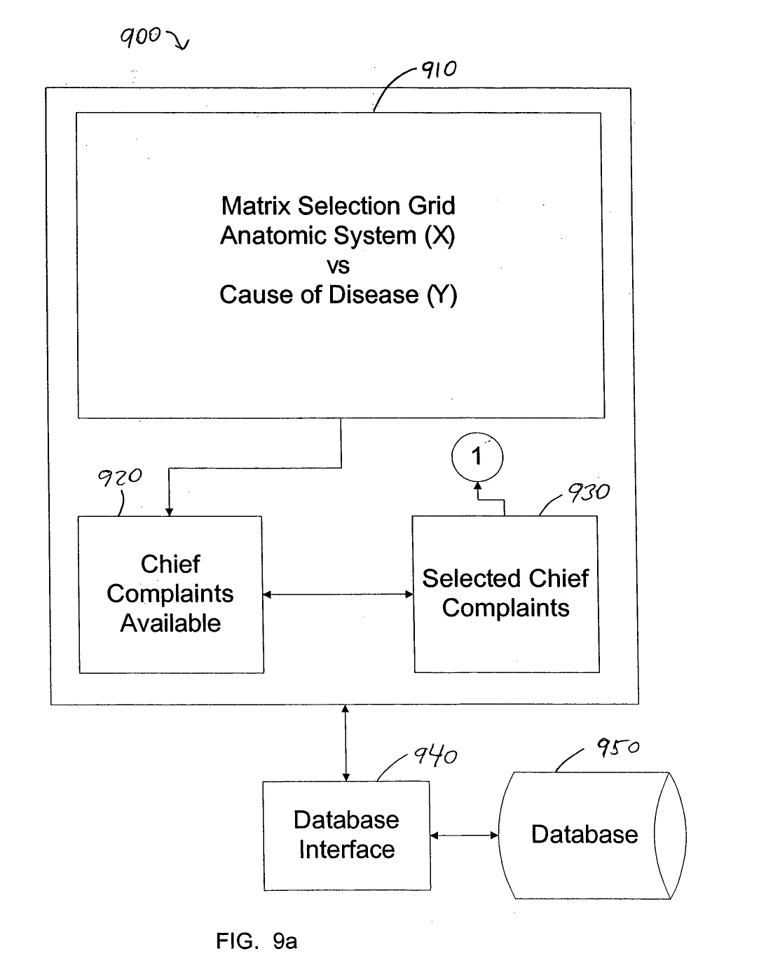

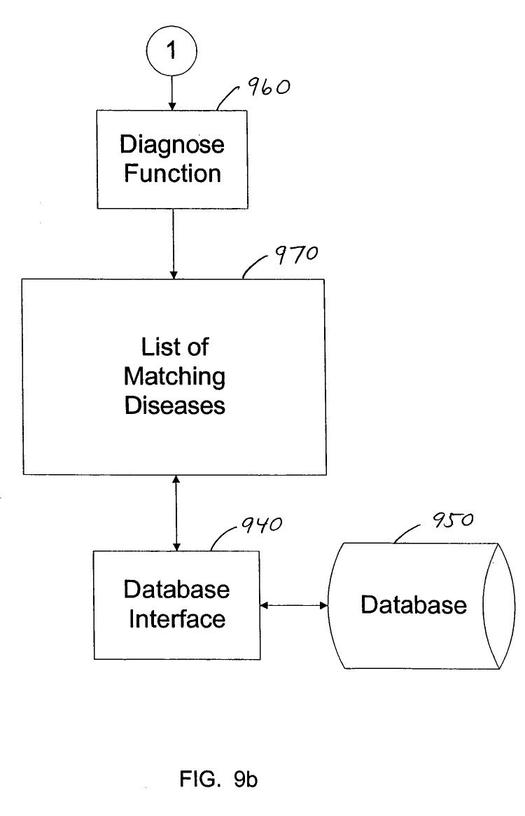

Referring to FIGS. 9a and 9b, a flowchart 900 of one embodiment of a process of operation for the system shown in FIG. 1 will be described. Beginning with a matrix selection grid 910 of anatomic system vs. cause of disease (such as shown in FIG. 2), a list of chief complaints 920 corresponding to the selection made by user on the grid is presented to the user. In one embodiment, a database 950 (which can be the database 140 (FIG. 1) or the database stored in storage 116) provides data to the matrix process via a database interface 940 (which can be interface 132 across the network 120 or an interface in computing device 110). In one embodiment, the user selects one or more symptoms or chief complaints 930 using the pick list function and selects 960 the diagnose button D (FIG. 6). In one embodiment, the diseases that match 970 are displayed with diseases that have both (all) chief complaints first, then n−1, etc.

In one embodiment, the matrix process shows the diseases matching all of the selections to one selection, e.g., from most hits to least hits. The symptoms, signs, etc. are actually weighted so the disease with the highest score is at the top of the list.

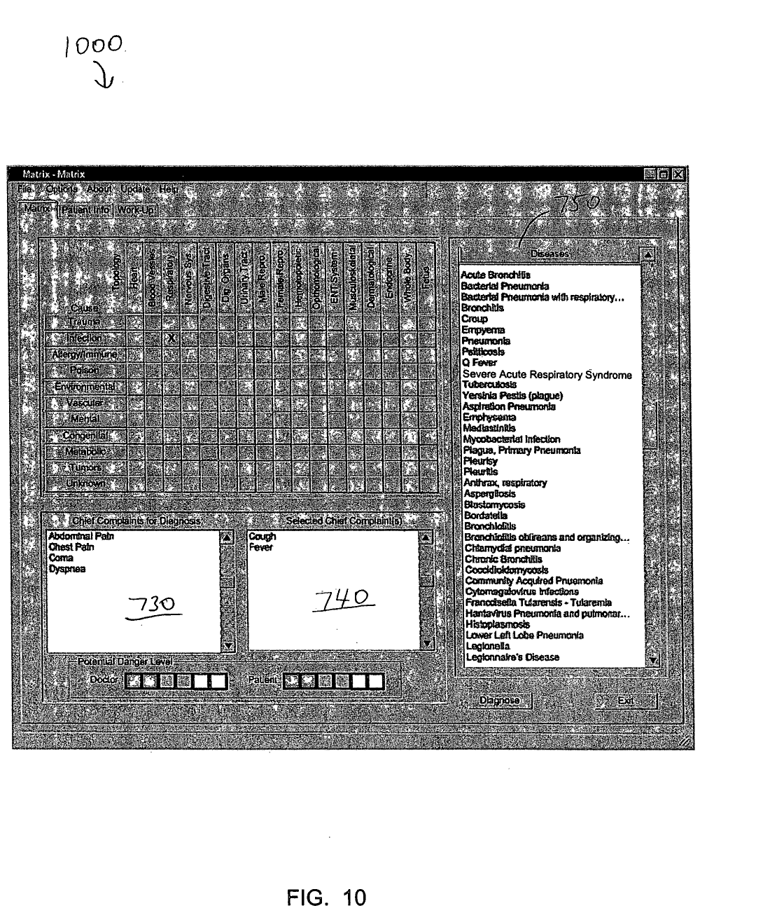

Referring to the example display 1000 shown in FIG. 10, from the chief complaint window 730, the selected Chief Complaints 740 of Cough and Fever yield a most likely match with Acute Bronchitis. Croup (from the previous example of FIG. 8) is demoted behind Pneumonia and Bronchitis as the most likely diagnosis in the list area 750.

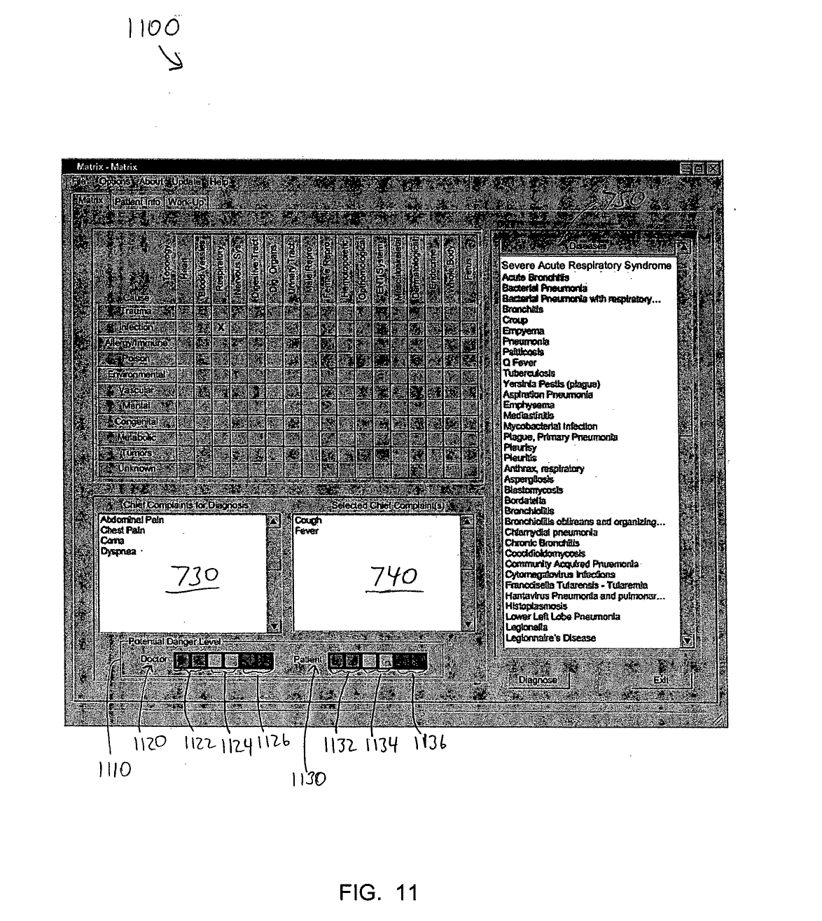

Once the DIAGNOSE button (area D of FIG. 6) is pressed or selected, the Matrix application 114 (FIG. 1) takes into account not only the Chief Complaints, but also patient specific information, in one embodiment. Referring back to FIG. 5, note that the patient is a female having an age in her 20's who has recently been to both Canada and China. This external bit of information is sufficient to re-value the most likely candidate from “Acute Bronchitis” to “Severe Adult Respiratory Syndrome”. Refer to the exemplary screen display 1100 of FIG. 11 to see the adjustment in the diagnosis window 750, as compared to the disease list of the screen display 1000 shown in FIG. 10.

Worry/Danger Indicators

Referring again to FIG. 11, a portion 1110 of the matrix tab having an indicator 1120 of the potential seriousness or danger level of the selected medical diagnoses to a healthcare worker and an indicator 1130 of the potential seriousness or danger level of the selected medical diagnoses to the patient will be described. In one embodiment, the indicator 1120 for the healthcare worker comprises several colored segments in a bar graph or thermometer analogue, such as two green segments 1122, two yellow segments 1124, and two red segments 1126. In this example, the more potential seriousness or danger of the selected medical diagnoses to a healthcare worker, the more segments are displayed. Similarly, the indicator 1130 for the patient comprises several colored segments in a bar graph, such as two green segments 1132, two yellow segments 1134, and two red segments 1136. In this example, the more potential seriousness or danger of the selected medical diagnoses to the patient, the more segments are displayed. The screen portion 1110 rapidly indicates and warns a less-sophisticated health care worker evaluating people in perhaps a third world country that there is significant danger to their health, e.g., such as for Ebola virus in Africa. Each disease has a field in the database having a relative value for how dangerous the disease is to the patient and the healthcare giver. In addition, the patient's age, sex and recent travel visits (geography) are also considered in the generation of the indicator values for the healthcare worker and the patient. Of course, other types of indicators, segmentation, colors and so forth are contemplated.

Spectrum of Sorts

Once the initial differential diagnosis is displayed, the user can sort this list by a number or parameters, for example, alphabetically, by age and gender, by the acuity of the disease, by the seriousness of the disease, etc. These include the following: incidence of disease by age, sex, race, travel history, risk factors, and so forth.

One of the important features of the Matrix application 114 (FIG. 1) is that a user can look at diseases (and symptoms, signs, etc.) from many different points of view. In fact, the user can customize the way the Matrix works by allowing the user to actually select any combination of fields in the database to sort on. This can be implemented by dragging and dropping criteria to the disease bar or by selecting the fields from a drop down list box.

Thus a clinician can look at the most probable cause of chills fever and sweats in a patient in Africa, for example.

Planning the Work-up

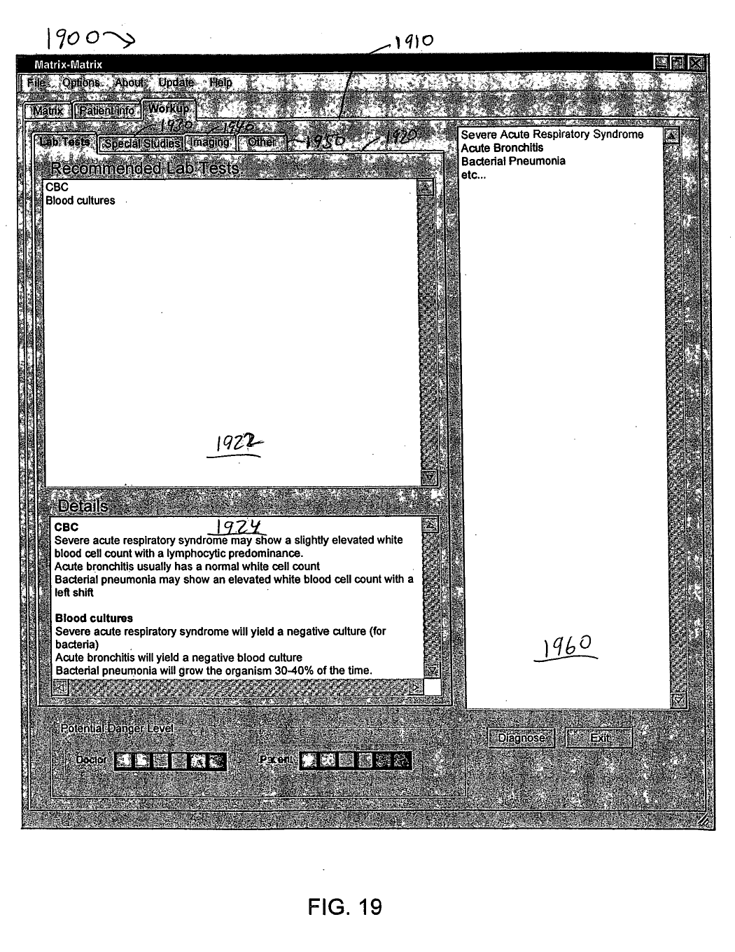

Referring to FIG. 19, an exemplary screen display 1900 for the matrix application of a work-up tab window 1910 having a lab tests tab 1920 selected will be described. In one embodiment, the work-up tab 1910 also includes lower level tabs for special studies 1930, imaging 1940, and other items 1950. The lab tests tab 1920 can include a recommended lab tests window 1922, where the recommended lab tests correspond to the top “n” diseases, e.g., three diseases, of the current differential diagnosis window 1960. Each disease has a field in the database for the laboratory test of choice, the special study of choice (e.g., EKG), the imaging modality of choice and the treatment of choice. The matrix application takes the top “n” diseases, looks up all of the “of choice” tables, discards the duplicate tests and lists them for the doctor or other medical caregiver. A details window 1924 provides further information about the recommended lab tests to the medical caregiver.

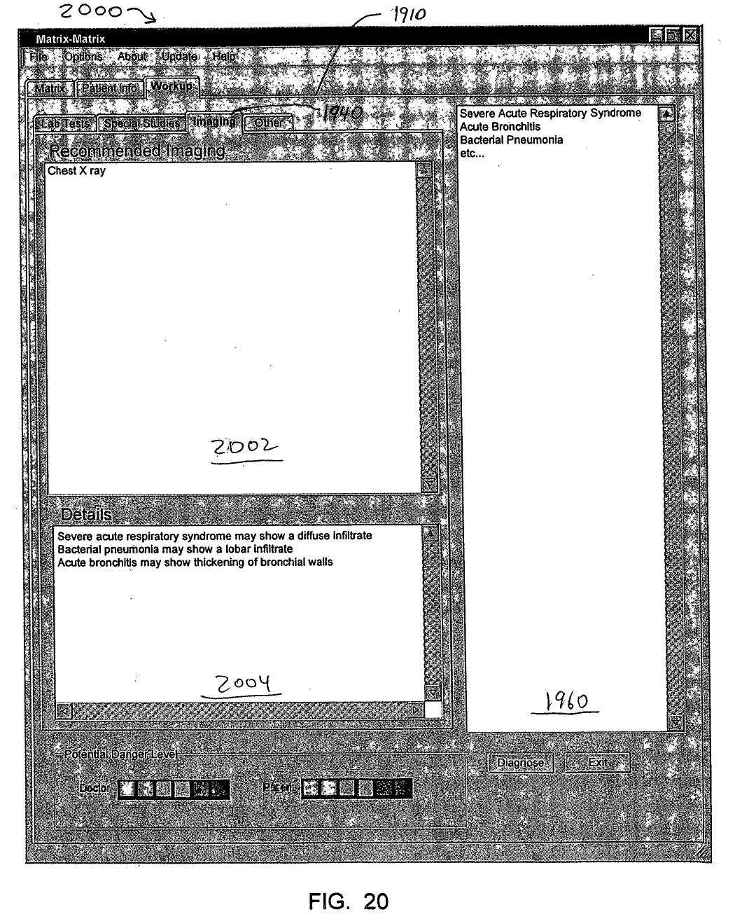

Referring to FIG. 20, an exemplary screen display 2000 for the matrix application of the work-up tab window 1910 having an imaging tab 1940 selected will be described. In one embodiment, the imaging tab 1940 can include a recommended imaging window 2002 listing the type(s) of recommended imaging to perform, and a details window 2004 providing further information about the recommended imaging to the medical caregiver. The recommended imaging corresponds to the top “n” diseases of the current differential diagnosis window 1960.

Operation

The Matrix application 114 (FIG. 1), operationally, is a convergence of well understood linear diagnostic processes tied together by both time and the relational nature of the data the Matrix is required to handle. In short, the aggregated process combines diagnosis as practiced by modem medicine, with the benefits of being able to draw on current topical knowledge in all medical disciplines.

Referring back to FIG. 1, the focus will be first on the computing device 110, and second on the data interface and storage portion of the diagram. This description of the operation is supported by additional detail diagrams that are described below.

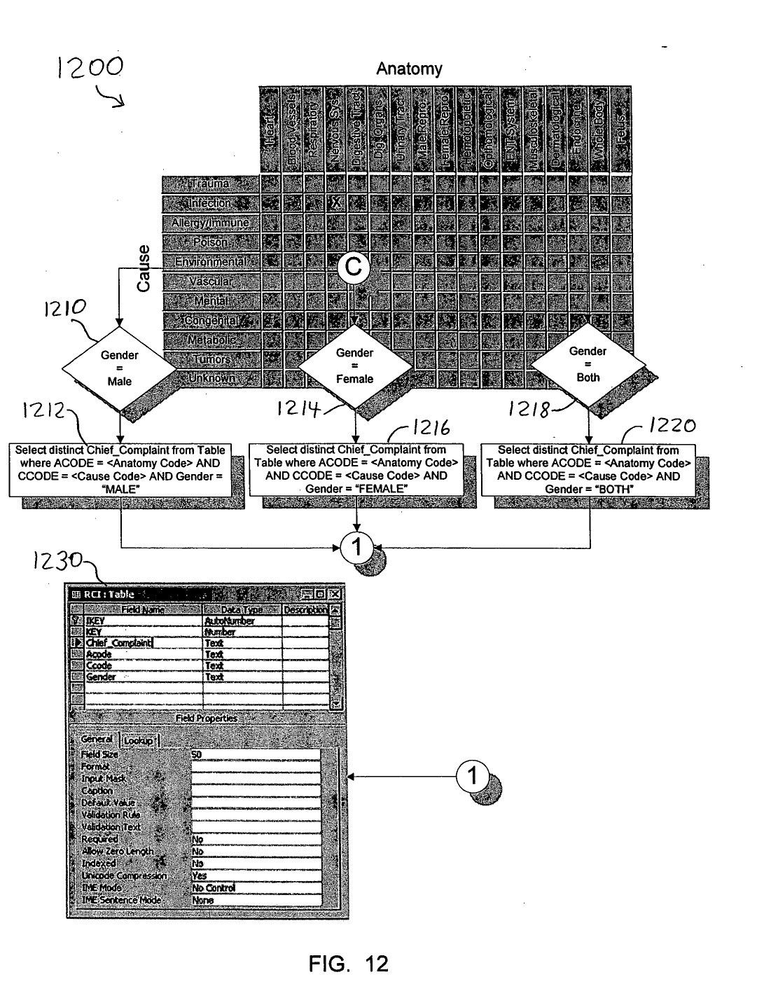

As demonstrated previously, the embodied Matrix screen provides a selection grid to begin the process of narrowing the possible solutions to the diagnosis. Referring to FIG. 12, a diagram illustrating exemplary operation 1200 of a portion of the matrix application after a cell on the array is selected will be described. In one embodiment, once the selection is made on the grid (area C), the application constructs a query 1212, 1216 or 1220 to a table in the database 1230 (either remote or local) that requests all Chief Complaints that fit the index selected, filtered by population specific information, e.g., gender=male 1210, gender=female 1214, or gender=both 1218, respectively. That list is then placed in the Chief Complaints window (e.g., area 730, FIG. 7) as an option to help further narrow the list of possible diagnostic solutions. In another embodiment, another attribute (as shown in FIG. 18) other than chief complaint can be utilized.

There are at least two variants to this operation, as described below.

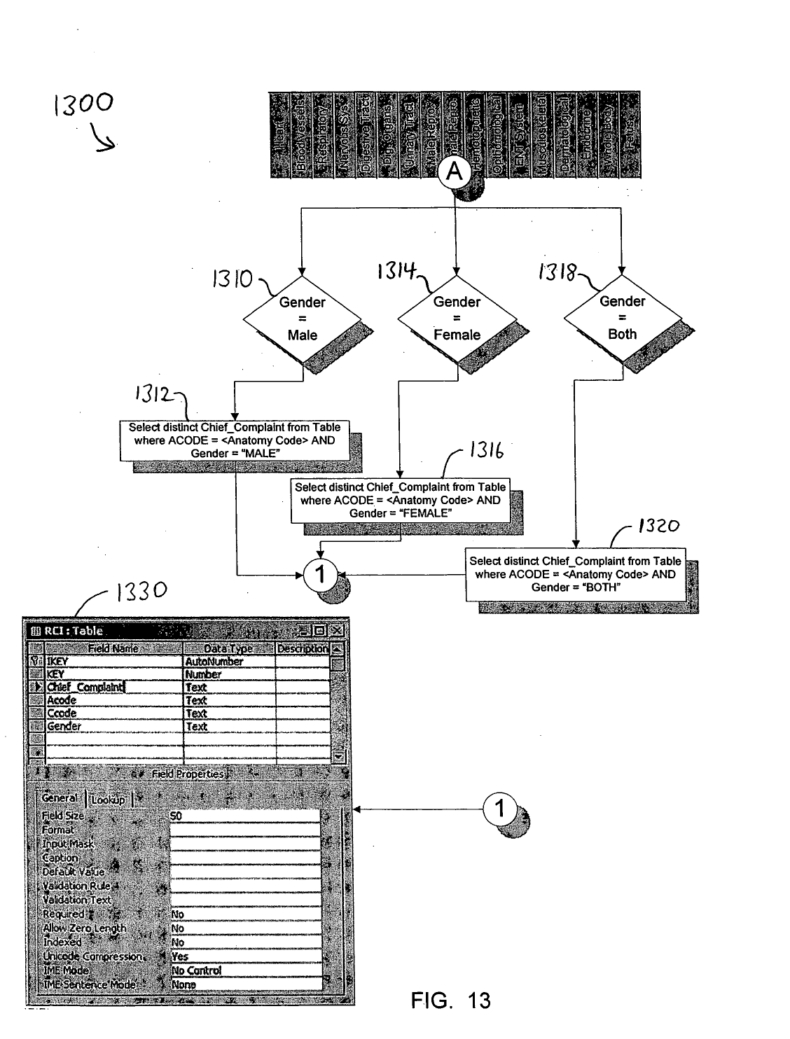

The first variation is that the physician has a good idea what anatomic system is affected, but not the cause. Referring to FIG. 13, a diagram illustrating exemplary operation 1300 of a portion of the matrix application after an anatomic system column on the array is selected will be described. In this case, the physician would select the anatomic system of interest in area A, by clicking on the anatomic label. The matrix application constructs a query 1312, 1316 or 1320 to the database 1330 (either remote or local) that requests all Chief Complaints associated with that particular anatomic system, filtered by population specific information, e.g., gender=male 1310, gender=female 1314, or gender=both 1318, respectively.

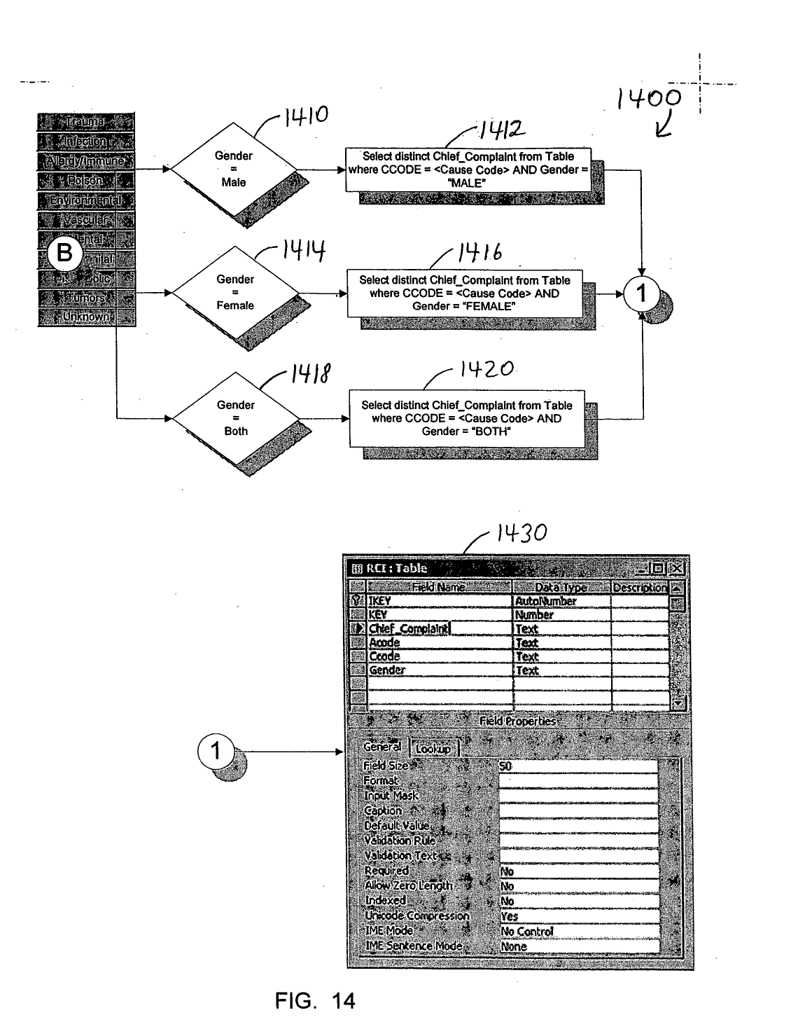

The second variation centers on a suspected or known cause, but it is indeterminate at the current moment as to which anatomic system(s) are affected. Referring to FIG. 14, a diagram illustrating exemplary operation 1400 of a portion of the matrix application after a cause row on the array is selected will be described. In this case, the physician would select the cause of interest in area B, by clicking on the cause label. The matrix application constructs a query 1412, 1416 or 1420 to the database 1430 (either remote or local) that requests all Chief Complaints associated with that particular cause, filtered by population specific information, e.g., gender=male 1410, gender=female 1414, or gender=both 1418, respectively.

In each of these instances where a cell, column, or row is selected in the array, the most likely disease candidates carry a “weight” that promotes them over their peers in the disease list, which leads to these disease candidates appearing toward the top of the disease list, in one embodiment. For example, see the disease list shown in FIG. 7. Furthermore, a list of applicable Chief Complaints is displayed in the Chief Complaints for Diagnosis window 730.

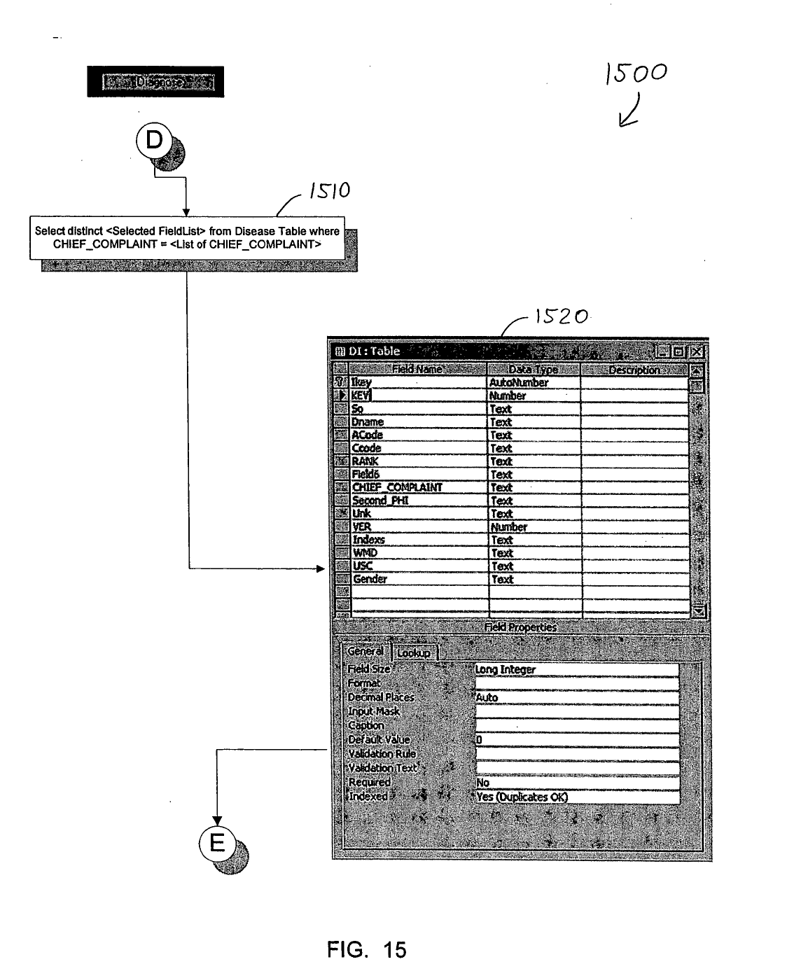

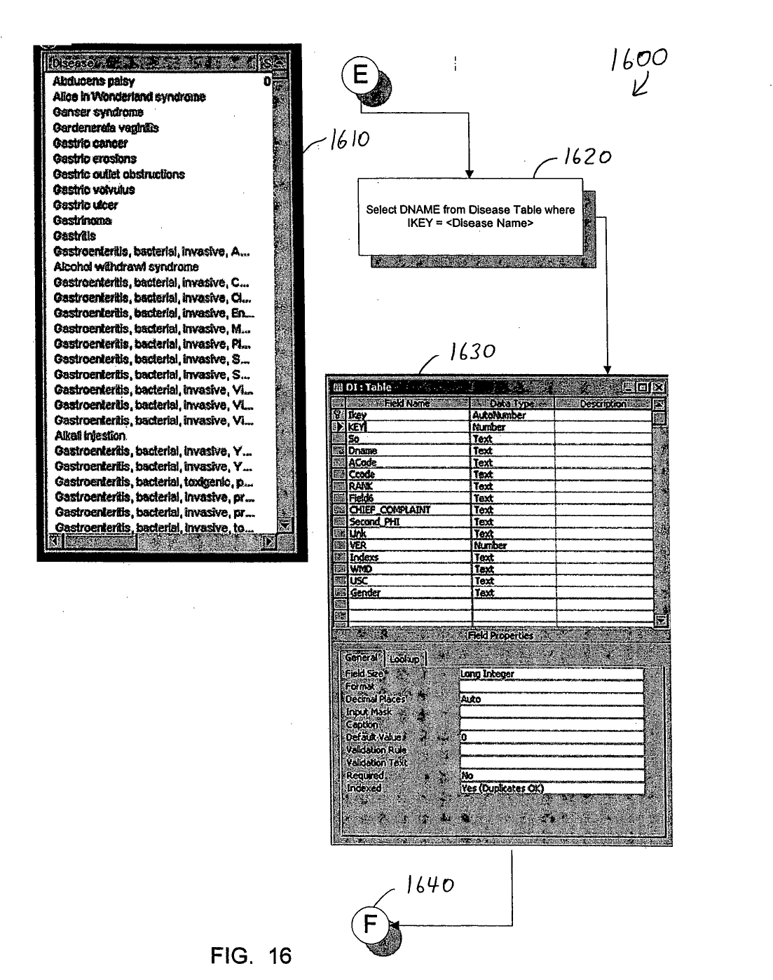

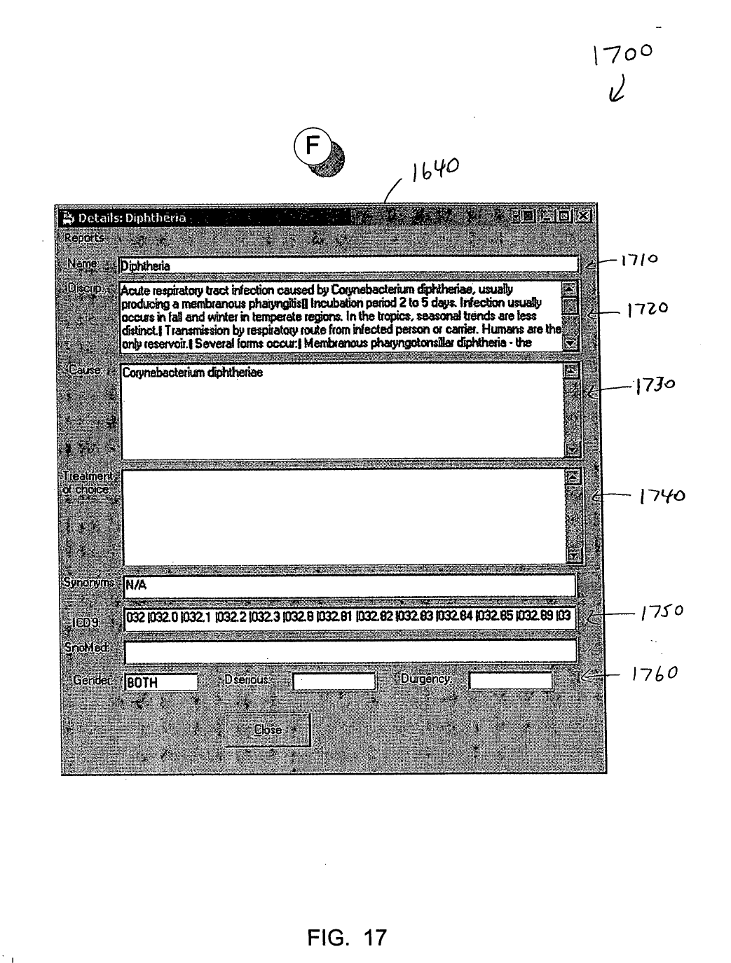

Once the final list of Chief Complaints 740 (FIG. 10) is selected by the physician, the physician can press the DIAGNOSE button D (FIG. 6) to launch the final filtering process of the disease list solution set. Referring to FIG. 15, a diagram illustrating exemplary operation 1500 of a portion of the matrix application after the diagnose button D is activated will be described. The matrix application creates a distinct data list based on the selected Chief Complaints as shown in the query 1510 of a disease table 1520 in the database. This list is the basis for creating the final filtered solution set of diseases for area E (FIG. 6). Referring to FIG. 16, a diagram illustrating exemplary operation 1600 of a portion of the matrix application after the diagnose button activated and data from the patient information tab (FIG. 5) is applied will be described. The matrix application takes the just created distinct disease data list 1610 of area E and applies that list, plus the external data, such as population, travel, ethnic, and gender information, to the creation of a query 1620 of the disease table 1630 in the database and selection of the final list and ranking of diseases that are displayed in the disease window 750 such as shown in FIG. 11. Upon scanning the final disease list, the physician selects what he or she believes to the disease of greatest interest. Referring to FIG. 17, an exemplary screen display 1700 of information about a particular disease selected from the ranked possible diagnoses will be described. A new window (F) 1640 presents a more detailed discussion of the disease 1710, its description 1720, possible and known causes 1730, treatment(s) of choice 1740, and other information relevant to the diagnosis and treatment of that disease, such as ICD9 (or other types) codes 1750, and gender, seriousness of the disease and urgency of the disease in area 1760.

Web Integration

Though it operates on a handheld computing device in one embodiment, the Matrix application may at any time connect to a web site where updates are available (see FIG. 1). This allows the system to download (and upload) new information about epidemic diseases (e.g., SARS), chemical and biological weapons agents, and so forth.

Further, patient specific information, specifically an electronic medical record (EMR), could be downloaded to the Matrix so that information already known about the patient would not have to be established a second time. Furthermore, in one embodiment, the EMR can be updated with or store the cause of disease, the anatomic system, at least one of the most likely candidate medical diseases or diagnoses, and/or the user-selected subset of the list of possible Chief Complaints. The EMR can be provided to a patient's physician, an emergency room physician, a patient's insurance company, and a managed health organization as desired or necessary. The structure and organization of EMRs are well known in the medical informatics community.

In another embodiment, the Matrix operates when connected to a global computer network and a central or distributed database via one or more servers having a database interface, such as SQL or ODBC.

Rural Areas

Very soon handhelds will find universal web access by way of networking protocols and the use of satellites for use in the most remote parts of the world. A caregiver equipped with the Matrix system would then be able to offer world class diagnosis to anyone anywhere in the world. Low cost simple handheld devices with satellite or network access can be an economic way of providing this service.

Summary of Certain Features of Embodiments

Layered format
Quick differential diagnosis
Best Workup
Pick List functionality
Updatability/Currency
Web-linked for updates
   Certain Benefits of Various System Embodiments

The Matrix allows physicians to quickly create a sophisticated, population and then patient-specific differential diagnosis for the patient's presenting complaints. In addition, it allows access to the way in which to differentiate between the final diagnostic list by planning the best and most efficient work up.

Description of the Matrix Main Screen Embodiment

Referring to FIG. 7, portions of the exemplary Matrix Main screen will be described for one embodiment of the application.

Disease List (Right Side)

The Disease List 750 shows all of the diseases in the database that match the latest user request. If the user last clicked a cause row, the Disease List shows all the diseases that the selected cause can produce. If the user last clicked the Diagnose Button, the diseases show the differential diagnosis for the Current Case.

| | |
|---|---|
| Single Click | [future action] |
| Double click | Lets user see and edit the disease elements |
| Right Click | Lets user to edit some disease |
| | fields like Age Range, Prevalence, |
| | Seriousness, and Urgency |

Anatomic Location of Disease (Columns)

The columns across the top of the Main Screen represent the anatomic location of a disease. At the top level, are the major anatomic systems and organs such as Cardiovascular, Respiratory, and Nervous. At lower levels, the columns represent anatomic components and elements.

At any level, a given column can be used to list either all the diseases of the given anatomic structure, or—by clicking an individual cell—to list only the diseases with a specific cause.

| | |
|---|---|
| Hover Mouse | Displays the meaning of the Anatomy Code. |
| Single Click | Lists all diseases in the |
| | database for the clicked column. If a |
| | column has no diseases in the |
| | database, the disease list is empty. |
| Double click | Details the anatomy for the |
| | clicked column. Thus Blood Vessels |
| | becomes Arteries, Aorta, Coronary, etc. |

Cause of Disease (Rows)

The rows down the left side of the Main Screen represent the major causes of disease such as Trauma, Infection, and Poisoning. A given row can be used to list all diseases caused by a single cause, or to show only the diseases caused in a specific anatomic system.

| | |
|---|---|
| Hover Mouse | Displays the meaning of the Cause Code. |
| Single Click | Lists all diseases in the database |
| | for the clicked row. If a cell has |
| | no diseases in the database, the disease list is empty. |
| Double Click | Lists sub-causes for the clicked row |

Cause and Anatomy (Cells)

The cells at the intersection of the rows and columns represent all of the diseases that a given cause can produce in a given anatomic structure.

| | |
|---|---|
| Single Click | Lists all diseases in the database |
| | for the selected row and column. |
| | If a cell has no diseases, the disease list is empty. |
| Double click | Details both the cause and |
| | anatomy for the clicked row and column |

Level Selector (Upper Left Corner)

| | |
|---|---|
| Single Click | Returns to the top level Matrix. Does |
| | nothing if the user is already the top level. |

Chief Complaint List (Bottom Left)

This is a list of chief complaints (CC) 730 for the diseases in the Disease List. Complaints can be selected and moved into the Current Case by clicking them.

| | |
|---|---|
| Single Click | Moves the clicked CC into the Current Case |

Current Case (Lower Center)

This is a list of complaints 740 that the user has so far compiled by selecting complaints from the Complaints List. Complaints can be moved back to the Complaint List by clicking them.

| | |
|---|---|
| Single Click | Deletes a complaint from the |
| | Current Case and moves it |
| | back to the Complaints List. |

Diagnose Button (Bottom Center)

| | |
|---|---|
| Single Click | Generates a differential diagnosis |
| | for the Current Case and |
| | displays it in the Disease List. |

Conclusion

Specific blocks, sections, devices, functions, processes and modules may have been set forth. However, a skilled technologist will realize that there are many ways to partition the system, and that there are many parts, components, processes, modules or functions that may be substituted for those listed above.

While the above detailed description has shown, described and pointed out the fundamental novel features of the invention as applied to various embodiments, it will be understood that various omissions and substitutions and changes in the form and details of the system illustrated may be made by those skilled in the art, without departing from the intent of the invention. The foregoing description details certain embodiments of the invention. It will be appreciated, however, that no matter how detailed the foregoing appears, the invention may be embodied in other specific forms without departing from its spirit or essential characteristics. The described embodiment is to be considered in all respects only as illustrative and not restrictive and the scope of the invention is, therefore, indicated by the appended claims rather than by the foregoing description. All changes which come within the meaning and range of equivalency of the claims are to be embraced within their scope.

1. A computerized system having a visual display for use in medical diagnosis, the system comprising:
a user interface operable to transmit data to the visual display of a computing device, the user interface configured to substantially simultaneously receive a plurality of identified attribute values representative of a medical condition of a patient; and
a data structure stored in a memory in data communication with a processor in the computing device, the data structure being configured, such that when accessed by the plurality of attribute values, to provide a list of one or more selected medical diagnoses of the patient for display via the user interface,
wherein the plurality of attributes includes at least a first attribute corresponding to a cause of disease and a second attribute corresponding to an anatomic system of the patient.

2. The system of claim 1, wherein the provided data is identified as being located within the data structure based on the plurality of attribute values.

3. The system of claim 1, wherein the memory is included in the computing device.

4. The system of claim 3, wherein the memory is removable.

5. The system of claim 1, wherein the memory is updated via data received from a global computing network.

6. The system of claim 1, wherein the computing device is portable.

7. The system of claim 1, wherein the computing device connects wirelessly to a global computing network.

8. The system of claim 1, wherein the user interface displays a representation of at least a portion of the data structure for a user.

9. The system of claim 8, wherein the representation includes a matrix having symbols indicative of the values of the first attribute along one axis of the matrix and symbols indicative of the values of the second attribute along the other axis of the matrix.

10. The system of claim 9, wherein the intersection of the identified attribute values on the matrix identifies the list of one or more selected medical diagnoses for the patient.

11. The system of claim 9, wherein the representation includes symbols indicative of a subclassification of the values of the first attribute and symbols indicative of a subclassification of the values of the second attribute when an input signal is received from the user.

12. The system of claim 9, wherein the intersection of the identified attribute values on the matrix identifies a list of one or more possible chief complaints for the patient.

13. The system of claim 1, wherein the list of one or more selected medical diagnoses for the patient includes a confidence level associated with each diagnosis.

14. The system of claim 13, wherein the list of one or more selected medical diagnoses for the patient comprises a rank-ordered differential diagnosis.

15. The system of claim 1, wherein the anatomic system attribute values include cardiovascular, respiratory, nervous, digestive/gastrointestinal, ear/nose/throat (ENT), ophthalmologic, gynecologic/obstetric, urologic, blood/hematological, skin/dermatological, bones/orthopedic, and endocrine.

16. The system of claim 1, wherein the cause of disease attribute values include allergy, environment, infection, mental, poison, trauma, vascular, genetic, nutritional/metabolic/endocrine, and tumor.

17. The system of claim 1, wherein each cause of disease is subdivided into subcauses of disease.

18. The system of claim 1, wherein each anatomic system is subdivided into anatomic subsystems.

19. The system of claim 9, wherein the intersection of the identified attribute values on the matrix identifies a list of one or more potential medical diagnoses for the patient and a list of one or more possible chief complaints for the patient.

20. The system of claim 19, wherein the list of one or more selected medical diagnoses for the patient is identified based on the list of one or more potential medical diagnoses for the patient and a user-selected subset of the list of possible chief complaints for the patient when an input signal is received from the user.

21. The system of claim 12, wherein the list of one or more selected medical diagnoses for the patient is identified based on a user-selected subset of the list of possible chief complaints for the patient and one or more criteria when an input signal is received from the user.

22. The system of claim 21, wherein one of the criteria comprises the gender of the patient.

23. The system of claim 21, wherein one of the criteria comprises the country or region of the country in which the patient resides.

24. The system of claim 21, wherein the list of one or more selected medical diagnoses for the patient is sorted based on the one or more criteria.

25. The system of claim 21, wherein one of the criteria comprises the age of the patient.

26. The system of claim 21, wherein one of the criteria comprises risk factors of the patient.

27. The system of claim 21, wherein one of the criteria comprises a travel history of the patient.

28. The system of claim 21, wherein one of the criteria comprises the race of the patient.

29. The system of claim 21, wherein one of the criteria comprises a seriousness of the disease of the patient.

30. The system of claim 21, wherein the criteria are dynamically changed.

31. The system of claim 1, wherein the data structure comprises a list of diseases of the human body.

32. The system of claim 1, wherein a user of the computing device comprises a physician or other health care professional.

33. The system of claim 1, additionally comprising an electronic medical record for the patient, the electronic medical record storing the cause of disease, the anatomic system and at least one of the one or more selected medical diagnoses.

34. The system of claim 33, wherein the electronic medical record is provided to at least one of a patient's physician, an emergency room physician, a patient's insurance company, and a managed health organization.

35. The system of claim 20, additionally comprising an electronic medical record for the patient, the electronic medical record storing the cause of disease, the anatomic system, at least one of the one or more selected medical diagnoses, and the user-selected subset of the list of possible chief complaints.

36. The system of claim 21, additionally comprising an electronic medical record for the patient, the electronic medical record storing the cause of disease, the anatomic system, at least one of the one or more selected medical diagnoses, and the user-selected subset of the list of possible chief complaints.

37. The system of claim 1, wherein the user interface displays a representation of an indicator of the potential seriousness or danger level of the selected medical diagnoses to a healthcare worker.

38. The system of claim 1, wherein the user interface displays a representation of an indicator of the potential seriousness or danger level of the selected medical diagnoses to the patient.

39. The system of claim 1, wherein the user interface displays a representation of a work-up based on the selected medical diagnoses.

40. The system of claim 39, wherein the work-up comprises a laboratory test of choice and an imaging modality of choice.

41. The system of claim 39, wherein the work-up comprises a special study of choice and a treatment of choice.

42. The system of claim 1, wherein the user interface displays a representation of at least one of a history, physical signs, tests, and treatments associated with the patient.

43. A computerized method used in medical diagnosis, the method comprising:
displaying a user interface on a computing device to a user;
corresponding a first attribute of a medical condition to a cause of disease and a second attribute of the medical condition to an anatomic system of a patient;
substantially simultaneously receiving a plurality of identified attribute values representative of the medical condition of the patient via the interface;
accessing, via the computing device, a data structure based on the plurality of attribute values; and
providing a list of one or more selected medical diagnoses associated with a diagnosis of the patient to the user interface based on the accessing.

44. The method of claim 43, additionally comprising storing the data structure in a memory of the computing device.

45. The method of claim 43, additionally comprising storing the data structure on a computing network having at least intermittent communication with the computing device.

46. The method of claim 43, additionally comprising providing the list of the one or more selected medical diagnoses for the patient from the data structure.

47. The method of claim 43, wherein displaying the user interface comprises displaying a representation of at least a portion of the data structure.

48. A computerized method used in medical diagnosis, the method comprising:
displaying a user interface on a computing device to a user;
substantially simultaneously receiving a plurality of identified attribute values representative of a medical condition of a patient via the interface, wherein a first attribute of the medical condition corresponds to a cause of disease and a second attribute of the medical condition corresponds to an anatomic system of the patient;
accessing, via the computing device, a data structure based on the plurality of attribute values; and
providing a list of one or more selected medical diagnoses associated with a diagnosis of the patient to the user interface based on the accessing.

49. A computerized medical diagnosis system, the system comprising:
means for displaying a user interface on a computing device to a user;
means for substantially simultaneously receiving a plurality of identified attribute values representative of a medical condition of a patient via the interface, wherein a first attribute of the medical condition corresponds to a cause of disease and a second attribute of the medical condition corresponds to an anatomic system of the patient;
means for accessing a data structure via the computing device based on the plurality of attribute values; and
means for providing a list of one or more selected medical diagnoses associated with a diagnosis of the patient to the user interface based on the accessing.

50. The system of claim 49, additionally comprising means for storing the data structure in a memory of the computing device.

51. The system of claim 49, additionally comprising means for storing the data structure on a computing network having at least intermittent communication with the computing device.

52. The system of claim 49, additionally comprising means for providing the list of the one or more selected medical diagnoses for the patient from the data structure.

53. The system of claim 49, wherein the means for displaying the user interface comprises means for displaying a representation of at least a portion of the data structure.
